(19) United States
(12) Patent Application Publication
(10) Pub. No.: US 2009/0292226 A1
(43) Pub. Date: Nov. 26, 2009

(54) MEDICAL SYSTEM AND METHOD EMPLOYING A DRUG DELIVERY ASSEMBLY

(75) Inventors: David Q. Feng, Mason, OH (US); Curt R. Eyster, West Chester, OH (US); Paul J. Niklewski, Cincinnati, OH (US)

Correspondence Address: THOMPSON HINE L.L.P.;Intellectual Property Group, P.O. BOX 8801, DAYTON, OH, 45401-8801, US

(73) Assignee: Ethicon Endo-Surgery, Inc., Cincinnati, OH (US)

(21) Appl. No.: 12/124,283

(22) Filed: May 21, 2008

(51) Int. Cl. A61M 21/00; A61B 5/11

(52) U.S. Cl. 600/595; 604/66

(57) ABSTRACT

A medical system includes a muscular-tension-measuring device and a drug delivery assembly. The muscular-tension-measuring device is operatively connectable to a patient and includes a device output having a device output signal which varies with involuntary changes in muscular tension of the patient. The drug delivery assembly is operatively connectable to the patient for controllably delivering at least one drug to the patient. In one implementation, the device output signal varies with involuntary changes in muscular tension of the patient caused by pain and/or anxiety. In one employment, drug delivery is varied by a controller or a user based at least on variations in the device output signal. A method for conscious sedation of a patient includes measuring muscular tension of the patient and includes varying delivery of a conscious sedation drug to the patient based at least on variations in the measured muscular tension of the patient.

### FIELD OF THE INVENTION

The present invention is related generally to medical technology, and more particularly to a medical system and to a medical method both employing a drug delivery assembly operatively connectable to a patient for controllably delivering at least one drug to the patient.

### BACKGROUND OF THE INVENTION

Known medical systems having drug delivery assemblies for controllably delivering at least one drug to the patient include sedation systems such as conscious sedation systems. A known conscious sedation system is disclosed in United States Patent Application Publication No. 2002/0017299. In that system, a controller generated a request for a predetermined response from a patient. The request was in the form of an auditory command which was received by a patient through an earphone in the ear of the patient or was in the form of a vibration signal which was received by the patient through a vibrator in a handpiece which was attached to the hand of the patient. The predetermined response to the request was the pushing of a button on the handpiece by the patient which closed a switch sending a signal to the controller. The controller analyzed medical information from the patient (such as blood pressure and other information) and analyzed the time delay between the request and the response to determine a level of sedation of the patient. When the time delay between the request and the response increased, the controller determined that the patient was in a deeper level of sedation and caused the drug delivery assembly of the conscious sedation system to decrease the flow of a conscious sedation drug to the patient.

Still, scientists and engineers continue to seek improved medical systems having a drug delivery assembly operatively connectable to a patient for controllably delivering at least one drug to the patient.

### SUMMARY

A first expression of an embodiment of the invention is for a medical system including a muscular-tension-measuring device and a drug delivery assembly. The muscular-tension-measuring device is operatively connectable to a patient and includes a device output having a device output signal which varies with involuntary changes in muscular tension of the patient when the device is operatively connected to the patient. The drug delivery assembly is operatively connectable to the patient for controllably delivering at least one drug to the patient. In one implementation, the device output signal varies with involuntary changes in muscular tension of the patient caused by pain experienced by the patient. In the same or a different implementation, the device output signal varies with involuntary changes in muscular tension of the patient in reaction to anxiety experienced by the patient.

A second expression of an embodiment of the invention is for a medical system including a muscular-tension-measuring device, a monitor, and a drug delivery assembly. The muscular-tension-measuring device is operatively connectable to a patient and includes a device output having a device output signal which varies with involuntary changes in muscular tension of the patient caused by pain experienced by the patient when the device is operatively connected to the patient. The monitor is connectable to the device output for providing a representation of the device output signal. The drug delivery assembly is operatively connectable to the patient for controllably delivering at least one drug to the patient. The drug delivery assembly is adapted to allow a user to vary the delivery of the at-least-one drug to the patient.

A third expression of an embodiment of the invention is for a medical system including a muscular-tension-measuring device, a drug delivery assembly, and a controller. The muscular-tension-measuring device is operatively connectable to a patient and includes a device output having a device output signal which varies with involuntary changes in muscular tension of the patient caused by pain experienced by the patient when the device is operatively connected to the patient. The drug delivery assembly is operatively connectable to the patient for controllably delivering at least one drug to the patient. The controller has a controller input operatively connectable to the device output and has a controller output operatively connectable to the assembly. The controller varies the delivering by the drug delivery assembly of the at-least-one drug to the patient based at least on variations in the device output signal.

A method of the invention is for consciously sedating a patient. The method includes measuring muscular tension of the patient and includes varying delivery of a conscious sedation drug to the patient based at least on variations in the measured muscular tension of the patient.

Several benefits and advantages are obtained from one or more of the expressions of the embodiment and the method of the invention. In one example, when the medical apparatus is used in a conscious sedation procedure such as a colonoscopy, a patient's combined level of pain and anxiety evident as muscular tension is measured for purposes of controlling the delivery of conscious sedation and analgesic drugs without requesting any response from the patient. This is of benefit to a patient who would have difficulty sensing requests and/or performing responses used in prior art response testing to determine the level of sedation of the patient.

### BRIEF DESCRIPTION OF THE FIGURES

FIG. 1 is schematic diagram of an embodiment of a medical system of the invention, wherein lines without reference numerals represent interconnecting cables; and

FIG. 2 is a graph of an example of the device output signal versus time of the device output of the muscular-tension-measuring device of the medical system of FIG. 1 which is representative of what might occur during a conscious-sedation medical procedure, wherein higher values of the device output signal would indicate increased levels of muscular tension in the patient corresponding to a decreased level of sedation and perhaps would call for an increase in the delivery of a conscious sedation and/or analgesic drug to the patient from the drug delivery assembly of the medical system of FIG. 1.

### DETAILED DESCRIPTION

Before explaining the expressions of an embodiment of the invention in detail, it should be noted that each is not limited in its application or use to the details of construction and arrangement of parts, instructions, and steps illustrated in the accompanying drawings and description. The illustrative embodiment of the invention may be implemented or incorporated in other embodiments, variations, and modifications, and may be practiced or carried out in various ways. Furthermore, unless otherwise indicated, the terminology employed herein has been chosen for the purpose of describing the illustrative expressions of the embodiment of the present invention for the convenience of the reader and are not for the purpose of limiting the invention.

It is further understood that any one or more of the following-described expressions of a medical system, implementations, etc. can be combined with any one or more of the other following-described expressions of a medical system, implementations, etc.

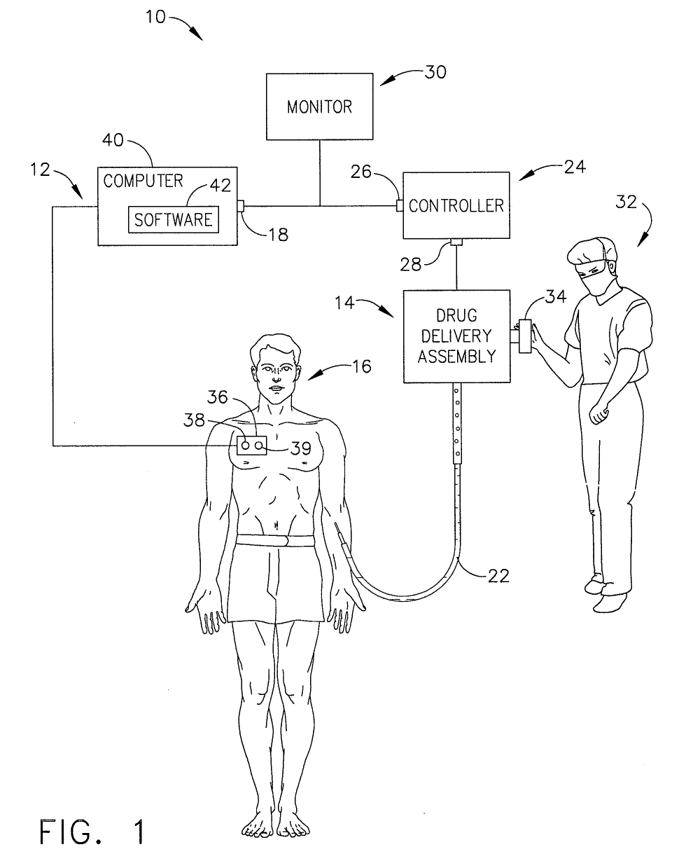

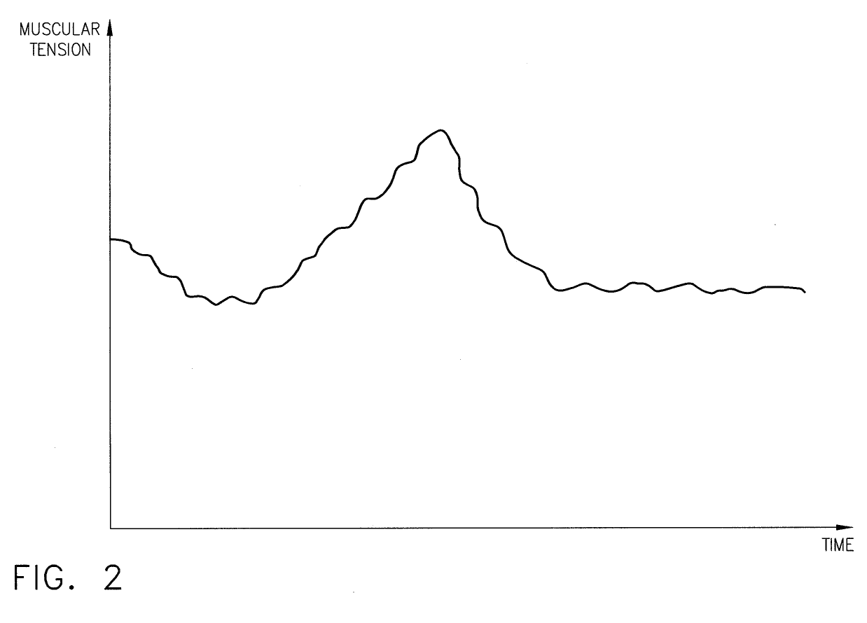

An embodiment of the invention is shown in FIG. 1. A first expression of the embodiment of FIG. 1 is for a medical system 10 including a muscular-tension-measuring device 12 and a drug delivery assembly 14. The muscular-tension-measuring device 12 is operatively connectable to a patient 16 and includes a device output 18 having a device output signal 20 (shown in FIG. 2) which varies with involuntary changes in muscular tension of the patient 16 when the device 12 is operatively connected to the patient 16. The drug delivery assembly 14 is operatively connectable to the patient 16 for controllably delivering at least one drug 22 to the patient 16.

In one arrangement of the first expression of the embodiment of FIG. 1, the assembly 14 delivers the at-least-one drug 22 into the patient 16. In one variation, intravenous (IV) drug delivery is employed using an IV pump. In a different variation, inhalation drug delivery using pressurized gas is employed by way of an oral and/or nasal cannula. Other variations are left to the artisan. In one example, the assembly 14 is operatively connected to the patient 16, and the device 12 is operatively connected to the patient 16.

In one implementation of the first expression of the embodiment of FIG. 1, the device output signal 20 varies with involuntary changes in muscular tension of the patient 16 caused by pain experienced by the patient 16. In the same or a different implementation, the device output signal 20 varies with involuntary changes in muscular tension of the patient 16 caused by anxiety experienced by the patient 16. In one example, the muscular-tension-measuring device 12 includes Myotest version 2.0 available from Myotest, Inc., 512 E. 11 Mile Road, Suite 100, Royal Oak, Mich. 48067. It is noted that Myotest version 2.0 will measure voluntary and involuntary changes in muscular tension. Myotest version 2.0 employs a sensor having an accelerometer and employs software running on a computer, wherein the sensor is connected to the computer, and wherein the computer output is a measure of muscular tension. In another example, the muscular-tension-measuring device includes surface electromyography, which is used in biofeedback, wherein a surface electrode sensor and filtering/processing software are used to measure muscle tension. Other choices for muscular-tension-measuring devices are left to those skilled in the art.

In one enablement of the first expression of the embodiment of FIG. 1, the medical system 10 also includes a controller 24 having a controller input 26 operatively connectable to the device output 18 and having a controller output 28 operatively connectable to the assembly 14, wherein the controller 24 varies the delivering by the assembly 14 of the at-least-one drug 22 to the patient 16 based at least on variations in the device output signal 20. In one example, other medical information from the patient 16, such as blood pressure, is also used by the controller 24 in addition to muscular tension.

In the same or a different enablement, the medical system 10 also includes a monitor 30 connectable to the device output 18 for providing a representation of the device output signal 20. In one variation, the monitor 30 provides a visual representation of the device output signal 20. In one example, the visual representation is similar to that of FIG. 2. In another example, the visual representation is a vertical bar graph whose height changes with changes in muscular tension. In the same or a different variation, the monitor 30 provides an audio representation of the device output signal 20. In one example, the audio representation is a tone which increase in loudness with increases in muscular tension. In another example, the audio representation is a tone which increases in pitch with increases in muscular tension.

In one application of the first expression of the embodiment of FIG. 1, the medical system 10 includes the monitor 30, and the assembly 14 is adapted to allow a user 32 to vary the delivery of the at-least-one drug 22 to the patient 16. In one example, the assembly 14 includes a knob 34 which controls the delivery of the at-least-one drug 22 to the patient 16. In another example, the assembly includes a touch screen (not shown) having icons allowing a user to vary the delivery of the at-least-one drug to the patient. In one variation, the knob 34 or touch screen allows the user to override the controller. In another variation, the controller is not present.

In one arrangement of the first expression of the embodiment of FIG. 1, the device 12 includes a muscular-tension sensor 36 which is disposable (and in one example is disposed) in contact with an area of skin of the patient 16. In one variation, the sensor 36 has at least one of an accelerometer 38 and an electrode 39. In one modification, the muscular-tension sensor 36 includes a computer 40 and software 42 running on the computer 40, wherein the sensor 36 is connected to the computer 40, and wherein the device output 18 is the computer output which is a measure of the muscular tension of the patient 16. It is noted that, in one example, the sensor 36 and the software 42 are the sensor and software of previously-discussed Myotest version 2.0 or of the previously-discussed surface electromyography. In one configuration, not shown, the computer 40, the monitor 30, and the controller 24 are integrated into a single unit.

In one employment of the first expression of the embodiment of FIG. 1, the at-least-one drug 22 has a sedative effect on the patient 16 and, in one example, is adapted for conscious sedation of the patient 16. In one variation, the controller 24 and/or the user 32 employs the at-least-one drug 22 for conscious sedation of the patient 16. In the same or a different employment, the at-least-one drug 22 has an analgesic effect on the patient 16. A drug having additional effects or other drugs having other effects are left to the artisan. It is noted that in the broadly-described first expression of the embodiment of FIG. 1, the at-least-one drug 22 can have any medical effect on the patient 16, such as having no sedating effect (e.g., a drug being administered to relax a tensed muscle) or having an anesthesia effect (wherein an increase in muscular tension indicates the patient is becoming conscious).

A second expression of the embodiment of FIG. 1 is for a medical system 10 including a muscular-tension-measuring device 12, a monitor 30, and a drug delivery assembly 14. The muscular-tension-measuring device 12 is operatively connectable to a patient 16 and includes a device output 18 having a device output signal 20 which varies with involuntary changes in muscular tension of the patient 16 caused by pain experienced by the patient 16 when the device 12 is operatively connected to the patient 16. The monitor 30 is connectable to the device output 18 for providing a representation of the device output signal 20. The drug delivery assembly 14 is operatively connectable to the patient 16 for controllably delivering at least one drug 22 to the patient 16. The drug delivery assembly 14 is adapted to allow a user 32 to vary the delivery of the at-least-one drug 22 to the patient 16.

In one example of the second expression of the embodiment of FIG. 1, the device 12 is operatively connected to the patient 16, the assembly 14 is operatively connected to the patient, and the monitor 30 is connected to the device output 18. In one arrangement, the device 12 includes a muscular-tension sensor 36 which is disposed in contact with an area of skin of the patient 16. In one variation, the sensor 36 has at least one of an accelerometer 38 and an electrode 39. It is noted that the arrangements, applications, etc. of the first expression of the embodiment of FIG. 1 are equally applicable to the second expression of the embodiment of FIG. 1.

A third expression of the embodiment of FIG. 1 is for a medical system 10 including a muscular-tension-measuring device 12, a drug delivery assembly 14, and a controller 24. The muscular-tension-measuring device 12 is operatively connectable to a patient 16 and includes a device output 18 having a device output signal 20 which varies with involuntary changes in muscular tension of the patient 16 caused by pain experienced by the patient 16 when the device 12 is operatively connected to the patient 16. The drug delivery assembly 14 is operatively connectable to the patient 16 for controllably delivering at least one drug 22 to the patient 16. The controller 24 has a controller input 26 operatively connectable to the device output 18 and has a controller output 28 operatively connectable to the assembly 14. The controller 24 varies the delivering by the drug delivery assembly 14 of the at-least-one drug 22 to the patient 16 based at least on variations in the device output signal 20.

It is noted that the examples, arrangements, etc. of the second expression and the arrangements, applications, etc. of the first expression of the embodiment of FIG. 1 are equally applicable to the third expression of the embodiment of FIG. 1.

A method of the invention is for consciously sedating a patient 16. The method includes measuring muscular tension of the patient 16 and includes varying delivery of a conscious sedation drug to the patient 16 based at least on variations in the measured muscular tension of the patient 16. It is noted that the examples, etc. of the first, second, and third expressions of the embodiment of FIG. 1 are equally applicable to the method.

Several benefits and advantages are obtained from one or more of the expressions of the embodiment and the method of the invention. In one example, when the medical apparatus is used in a conscious sedation procedure such as a colonoscopy, a patient's combined level of pain and anxiety evident as muscular tension is measured for purposes of controlling the delivery of conscious sedation and analgesic drugs without requesting any response from the patient. This is of benefit to a patient who would have difficulty sensing requests and/or performing responses used in prior art response testing to determine the level of sedation of the patient.

While the present invention has been illustrated by several expressions of a method, an embodiment, and enablements, applications, etc. thereof, it is not the intention of the applicant to restrict or limit the spirit and scope of the appended claims to such detail. Numerous other variations, changes, and substitutions will occur to those skilled in the art without departing from the scope of the invention. It will be understood that the foregoing description is provided by way of example, and that other modifications may occur to those skilled in the art without departing from the scope and spirit of the appended Claims.

What is claimed is:

1. A medical system comprising:
a) a muscular-tension-measuring device operatively connectable to a patient and including a device output having a device output signal which varies with involuntary changes in muscular tension of the patient when the device is operatively connected to the patient; and
b) a drug delivery assembly operatively connectable to the patient for controllably delivering at least one drug to the patient.

2. The medical system of claim 1, wherein the device output signal varies with involuntary changes in muscular tension of the patient caused by pain experienced by the patient.

3. The medical system of claim 1, wherein the device output signal varies with involuntary changes in muscular tension of the patient caused by anxiety experienced by the patient.

4. The medical system of claim 3, wherein the device output signal varies with involuntary changes in muscular tension of the patient caused by pain experienced by the patient.

5. The medical system of claim 1, also including a controller having a controller input operatively connectable to the device output and having a controller output operatively connectable to the assembly, wherein the controller varies the delivering by the assembly of the at-least-one drug to the patient based at least on variations in the device output signal.

6. The medical system of claim 1, also including a monitor connectable to the device output which outputs a representation of the device output signal.

7. The medical system of claim 6, wherein the monitor provides a visual representation of the device output signal.

8. The medical system of claim 6, wherein the monitor provides an audio representation of the device output signal.

9. The medical system of claim 8, wherein the monitor provides a visual representation of the device output signal.

10. The medical system of claim 1, wherein the assembly is adapted to allow a user to vary the delivery of the at-least-one drug to the patient.

11. The medical system of claim 1, wherein the device includes a muscular-tension sensor which is disposable in contact with an area of skin of the patient, wherein the sensor includes at least one of an accelerometer and an electrode.

12. The medical system of claim 1, wherein the at-least-one drug has a sedative effect on the patient.

13. The medical system of claim 13, wherein the at-least-one drug is adapted for conscious sedation of the patient.

14. The medical system of claim 1, wherein the at-least-one drug has an analgesic effect on the patient.

15. A medical system comprising:
a) a muscular-tension-measuring device operatively connectable to a patient and including a device output having a device output signal which varies with involuntary changes in muscular tension of the patient caused by pain experienced by the patient when the device is operatively connected to the patient;
b) a monitor connectable to the device output for providing a representation of the device output signal; and
c) a drug delivery assembly operatively connectable to the patient for controllably delivering at least one drug to the patient, wherein the assembly is adapted to allow a user to vary the delivery of the at-least-one drug to the patient.

16. The medical system of claim 15, wherein the device is operatively connected to the patient, the assembly is operatively connected to the patient, and the monitor is connected to the device output.

17. The medical system of claim 16, wherein the device includes a muscular-tension sensor which is disposed in contact with an area of skin of the patient, wherein the sensor includes at least one of an accelerometer and an electrode.

18. A medical system comprising:
a) a muscular-tension-measuring device operatively connectable to a patient and including a device output having a device output signal which varies with involuntary changes in muscular tension of the patient caused by pain experienced by the patient when the device is operatively connected to the patient;
b) a drug delivery assembly operatively connectable to the patient for controllably delivering at least one drug to the patient; and
c) a controller having a controller input operatively connectable to the device output and having a controller output operatively connectable to the assembly, wherein the controller varies the delivering by the assembly of the at-least-one drug to the patient based at least on variations in the device output signal.

19. The medical system of claim 18, wherein the device is operatively connected to the patient, the assembly is operatively connected to the patient, the controller input is operatively connected to the device output, and the controller output is operatively connected to the assembly.

20. The medical system of claim 19, wherein the device includes a muscular-tension sensor which is disposed in contact with an area of skin of the patient, wherein the sensor includes at least one of an accelerometer and an electrode.

21. A method for consciously sedating a patient comprising measuring muscular tension of the patient and varying delivery of a conscious sedation drug to the patient based at least on variations in the measured muscular tension of the patient.
